(19) United States
(12) Patent Application Publication
(10) Pub. No.: US 2003/0109490 A1
(43) Pub. Date: Jun. 12, 2003

(54) Composite stimulating iNOS enzyme which induce immuno-reactant nitric oxide synthesis and process for preparing the same

(76) Inventors: Hyung Ja You, Kyungki-do (KR); Sang Bong Seo, Kyungki-do (KR); Chan Seok Seo, Kyungki-do (KR)

Correspondence Address: PENNIE AND EDMONDS, 1155 AVENUE OF THE AMERICAS, NEW YORK, NY, 100362711

(21) Appl. No.: 10/192,804

(22) Filed: Jul. 9, 2002

(30) Foreign Application Priority Data
Jul. 9, 2001 (KR) 2001-40955

(51) Int. Cl. C12P 19/26; A61K 31/722

(52) U.S. Cl. 514/55; 435/84

(57) ABSTRACT

The present invention relates to a composite stimulating iNOS enzyme which induce immuno-reactant Nitric Oxide (NO) synthesis and a process for preparing the same. More particularly, it relates to a composite stimulating iNOS enzyme which is inducible immuno-reactant NO synthase prepared by nano-coating and binding water soluble β-glucosamin with immuno-protein where the water soluble β-glucosamin is produced by ultrasonic degradation of chitin/chitosan in NaCl solution, degradation employing lysozyme, washing with ethanol, and ion-exchange, thus containing distinctive functional element from the conventional chitosan.

### FIELD OF THE INVENTION

The present invention relates to a composite stimulating iNOS enzyme which induce immuno-reactant Nitric Oxide (NO) synthesis and a process for preparing the same. More particularly, it relates to a composite stimulating iNOS enzyme which is inducible immuno-reactant NO synthase prepared by nano-coating and binding water soluble β-glucosamin with immuno-protein where the water soluble β-glucosamin is produced by ultrasonic degradation of chitin/chitosan in NaCl solution, degradation employing lysozyme, washing with ethanol, and ion-exchange, thus containing distinctive functional element from the conventional chitosan.

### BACKGROUND OF THE INVENTION

Fiber and natural biomaterial is a complex composite of substances which is a high molecular weight compound having greater than 100,000 Da of a molecular weight and represents the content of substances that cannot be digested by the human digestive enzymes. Fiber is divided into two categories, water soluble and water insoluble. Even though the water soluble fiber provides more benefits compared to the water insoluble fiber, numbers of known water soluble fibers are very limited, thus the demand is therefore highly increased.

Of fibers, dietary fibers can improve the movement of food/fluid through the intestinal tract, strengthen immunity pharmacologically, lower cholesterol level, prevent cardiovascular disease, and be used as diet because it delays nutrient absorption. Further, it obstructs fat absorption and slows down absorption of glucose (sugar), thus being effective for diabetes. Therefore, the value of the dietary fibers will be increased as natural products when functionalities for biological properties are more investigated. Dietary fiber comes mostly from of the plant products but glucosamin fiber comes from the animal products of which structure is a little different from that of cellulose. Pectin, collagen and a few derivatives are the only water insoluble dietary fibers treated for being water soluble.

Therefore, it must be good news to separate and produce a composite stimulating iNOS enzyme based on hydrophilic β-glucosamin dietary fiber in a large quantity in order to utilize widely from a lack of proper materials in food and medicinal industries and allow the use of natural materials instead of synthetic materials mostly used in the modern society.

Particularly, chitin is one of the most abundant polysaccharides found in nature such as the shell of crustaceans like crap and shrimp. It has a chemical structure similar to that of cellulose but is an animal dietary fiber having higher than 1,000,000 Da of molecular weight. The difference between cellulose and chitin is that the 2-hydroxy group of the cellulose has been replaced with an acetamide group. This results in several 1→4 β-D glucopyranose structural units.

Chitosan, one of the most abundant polysaccharides, is an all natural product which is derived from deacetylation of chitin. It is a biomass having various biological properties such as anticancer activity, antibacterial function, cholesterol regulating action, stimulation of immune system, blood pressure regulating action, blood sugar regulating action and the like. However, the use of chitosan has been limited due to its poor solubility and poor reactivity. Immune activating carrier of the present invention will promise for the future in biotechnology field where natural materials and high molecular weight materials are insufficient. Chitin/chitosan are environmentally friendly materials as well as natural materials, thus they can be utilized widely and synergistically with development of their functional properties. However, they have been used meagerly in very limited fields because the reactivity is so little.

There has been no other immune activating carrier except chitin/chitosan. Hydrogen chloride salt of chitooligosaccharide as a water soluble chitosan derivative has been used only in the very limited area. Applications as quality precision products have not been introduced so far, thus development of novel natural dietary fibers having excellent desired efficacy to be suitable those applications is highly demanded.

Deficiency of dietary fibers invites a variety of diseases and particularly, human living in the modern society has a problem in immunity due to environment changes and immuno deficiency. Therefore, dietary fibers having excellent efficiency and self-immnunostimulatory activity may help essentially to prevent and cure many of diseases prevalent in modern society. Further, since natural materials which are to be excellent in naturally increasing functions are harmless and non-toxin to human body, they will be considered to be more valuable.

### SUMMARY OF THE INVENTION

As a result, the present invention has completed with the production of a composite stimulating iNOS enzyme which induces immuno-reactant NO synthesis by producing water soluble β-glucosamin fiber prepared by treatments of ultrasonic separation of chitin/chitosan in NaCl solution, separation with Lysozyme, washing with ethanol, ion exchange, and nano-coating and bonding with immuno-protein, which provides distinctive functional elements from the conventional chitosan.

Accordingly, an object of the present invention is to provide a composite stimulating iNOS enzyme which induces immuno-reactant NO synthesis and has its own characteristics of chitin/chitosan. Another object of the present invention is to provide a process for preparing the same.

### BRIEF DESCRIPTION OF DRAWINGS

The above and other objects and features of the present invention will become apparent from the following description of the invention, when taken in conjunction with the accompanying drawing, in which:

FIG. 1 is a diagram of composite stimulating iNOS according to the present invention;

FIG. 2 shows FT-IR charts of water soluble chitosan and non water soluble chitosan;

FIG. 3 is a graph showing the production of NO by N^{G}MMA;

FIG. 4 is a western blot of iNOS expression;

FIG. 5 is a western blot of TNF-α expression;

FIG. 6 shows activity of induced NF-kB from macrophage by the composite of the present invention;

FIG. 7 shows picture demonstrating acceleratory effect on the wound-healing process by cell regeneration of the composite of the present invention; and

FIG. 8 is a graph showing delivery of the composite into tissues.

### DETAILED DESCRIPTION OF THE INVENTION

The present invention provides a composite stimulating iNOS enzyme which induce immouno-reactant nitric oxide synthesis and a process for preparing the same.

The present invention is described in detail hereunder. The present invention provides a composite stimulating iNOS enzyme which is inducible NO synthease of macrophage having excellent properties and native characteristics of chitin, which provides unlimited potentialities in food and medicinal industries and further contribution to development of raw materials and human health.

The composite stimulating iNOS enzyme, which is inducible immuno-reactant NO synthetase, has properties of moisturizing ability by moving small and large intestines, ion exchange ability, gel formation, and infected portion- and wound-healing activities as well as properties which glucosamin has. Undesirable living and diet habits can bring chronic tissues and blood vessels having latent wide-spread inflammatory cells and even destroy tissues gradually. Particularly, it is noticed that destroyed small intestine tissues can cause of inflammation and diseases. The small intestine absorbs virtually all nutrients having small molecular weight. Bacteria and toxic substances in the large intestine can travel backwards because of bacteria, microbes, and toxic substances passing mucous membrane of the destroyed small intestine and malfunction of internal organs. Toxic substances, bacteria and high molecular weight compounds can be penetrated and absorbed to the small intestine. Generally, these toxic substances through the small intestine can be detoxified almost 100% in the normal liver. However, undesired living and diet habits, stress, environmental pollution and the like are main causes of lowering liver functions. The malfunctioning liver cannot perform complete metabolism, detoxify and clean properly and prevent from infections so that the possibility of getting diseases becomes high.

Immune activating carrier of the present invention has water soluble high molecular weight mucopolysaccharide constitution having a close correlation with immune infection routes.

First, the mucous membrane having moisturizing ability and regulating functions of mucopolysaccharides is formed in stomach, small intestine, and large intestine which are infection routes passing the digestive system. Immune activating composite can have preventive effects from the first infectious microorganism, harmful microorganism and pathogenic bacteria by inducing activities of adhesion of harmful microorganism or pathogenic bacteria, destroying cell membrane of harmful microorganism and pathogenic bacteria, and elute of cytoplasm. Second, the composite of the present invention preoccupies exclusively infection routes of harmful microorganism and pathogenic bacteria and is then absorbed into the blood and tissue of immune cells, and particularly activates macrophage which has significant immune activity at the foremost front of immune mechanism to block harmful microorganism and pathogenic bacteria.

Accordingly, immune activating composite of the present invention is considered as an excellent resource for alternative natural medicines and nutraceuticals to prevent diseases, strengthen self-immune system against many types of diseases, and cure diseases as well as prevent the secondary infection for the future.

Especially, since it is involved in biological mechanism to induce signals between cells so that NO synthesis in leucocytes is excellent, it provides valuable functions related to human health.

The immune activating composite of the present invention can be used as alternative medicines, natural materials, biomaterials, diet products, functional beverages, nutraceuticals, functional supplement foods, food additives, medicinal ingredients, additives, analgesics, materials for medicinal treatment, medicines for animals, living supplies, and the like and applied widely in the whole fields such as chemical engineering, cosmetic, and medicinal industries. Further, it has added valuable functions which were deficient in the conventional dietary fibers. The immune activating composite of the present invention is also harmless to human body because it is produced by NaCl, ultrasonic, physical, and fractional ion-exchange separation methods instead of chemical separation method. In addition, the immune activating composite is a significant natural high molecular weight fiber resource for the life science and bio-science industries which resolve chemical and environmental toxin problems. High molecular weight medicines having excellent permeation into blood vessels can be used in effectively drug delivery by controlling concentration in the body. The immune activating carrier immnunostimulatory carrier of the present invention has a molecular weight between 100,000 Da-1,000,000 Da and fine particles. It has excellent changes in the human body such as stimulation, diffusion, osmosis, and recognition and further has durability, cell modulating function, biocompatibility, stability and the like, thus being suitable fur novel materials for the future.

The following analyses and evaluations are performed to prove these values and advantages of the composite of the present invention. It is freeze dried to maintain efficiencies and stabilize the preparations; analgesic function, absorption and permeability into the blood is enhanced by controlling particle size and electric charge to improve blood circulation; and the composite has stability and compatibility.

The composite of the present invention permeates to the body by controlling the relations properly between affinity and repellent force to recognize cells and activate mRNA, thus it accelerates iNOS enzyme expression. The functional groups interacts with moisture in the body to draw hydrogen bonds with NH₃⁻ so that it may optimize transfer of target cells and carrying of high molecular weight compounds.

iNOS stimulating composite has a carbohydrate structure having added functional groups. The functions of carbohydrates have close relations with growth, bind, pinocytosis, antigen, fertilization, and differentiation. Especially, the immunostimulatory carrier of the present invention compounding β-glucosamin has glucose-amine-alcohol constitution having high molecular weight and containing 80% of amino group (NH₂). After saccharification of protein, the composite of the present invention has superior distinct functions and activities to perform its biological duties by controlling the final structure and functions of protein in the cell as post-translation modification. During the saccharification, high molecular weight water soluble materials make more progress and have stronger interaction compared to low molecular weight materials. It has been highly demanded to develop and use natural materials since human beings arc losing self-protection of health due to environmental pollution, overusing chemical drugs such as antibiotics. The immunostimulatory high molecular weight hydrophilic fiber of the present invention is natural material and has high prestige and high efficiency, thus being applicable in the medicinal and bio-science fields such as functional foods, additive materials, alternative natural medicines/medicinal ingredients, biomaterials, and the like.

According to the present invention, a fiber having water soluble β-glucosamin structure is prepared by performing ultrasonic degradation of water soluble chitosan in 10-45% of NaCl solution at pH 4.5-8.5 at a temperature of 10-80° C. and a wavelength of 10-80 KHz for 1-14 hours; degradation with lysozyme; washing with ethanol; fractional separation using cation-exchange treatment to obtain 1.1-1.9 polydispersity efficiency for the desired molecular weight fractions; ion-exchange more than once; and introduction of functional group. And then, the composite stimulating iNOS enzyme is prepared by nano-coating and binding the obtained fiber having water soluble β-glucosamin structure with 0.01-15% of immuno-protein using 0.01-2% of cholic acid preparation solution containing 0.001-15% of collagen as a catalyst and freeze-drying. The result composite was evaluated with RI detector and analysis of β-glucosamin content and finally produced. The iNOS enzyme stimulating composite of the present invention has basic glucose-amine-alcohol structure of which amino acid portion provides functional activities and glucose portion assists greatly in absorption, growing of amino acids and improving immunity.

Pharmaceutically acceptable carriers or additives as manufacturing agent into solid, liquid, and gel may be arbitrarily incorporated to prepare in the form of tablets, granules, capsules, suspensions, emulsions or preparations for parenteral administration which can be administered once or several times. Further, the composite stimulating iNOS enzyme of the present invention can be widely used as natural biomaterials such as drug absorption promoters, functional foods for preventing future diseases, alternative medicines, intravenous preparations, therapeutic agents, cell cleaners, regulating agents, drug carrier, therapeutic promoters, and the like.

For administration, dosages of the composite will generally be in the range of 1-300 mg daily for an average adult (70 Kg) in single or multiple doses, once or several times per day but can be varied with age, physical condition, weight and sex. A fatal dose is 3,000 mg/Kg.

The following Examples are intended to further illustrate the present invention without limiting its scope.

### EXAMPLE 1

### Preparation of a Composite Stimulating iNOS Enzyme which Induce Immuno-Reactant NO Synthesis

1000 g of water soluble chitosan having an average molecular weight of 100,000-500,000, which is prepared by following the procedure disclosed in U.S. Pat. No. 5,730,876, in 30% of NaCl solution was adjusted to be pH 6 to 7, and performed for ultrasonic degradation for 3 hours at 45° C. and 50 KHz of wavelength, followed by degradation with lysozyme. It was performed for ion exchange after standing at 80° C. for 2 hours to stabilize as well as prevent from transformation and change of NH₂ link. At this time, cationic resin, DIAION PK228 (SamYang Co., Korea) was passed at a rate of 1500 cc/min and anionic resin (Dupont Co., USA) was at a rate of 500 cc/min. Fractional separation using carbon filter to remove un-reacted materials, impurities, and remaining ions. Polydispersity efficiency of the desired molecular weight fractions was 1.1 to 1.9. It was noted that dissociated ions did not exist by checking cations and anions in the aqueous solution.

The solution composite was fed to the filtrations one after another having 1,000,000, 600,000, 300,000, 200,000, and 100,000 to remove remaining enzymes and separate compounds. The filtration device and filter having 200 mm by 300 mm (200 mm wide and 300 mm long) was prepared ourselves and yield was 95% (±10%) and purity was 7-99%

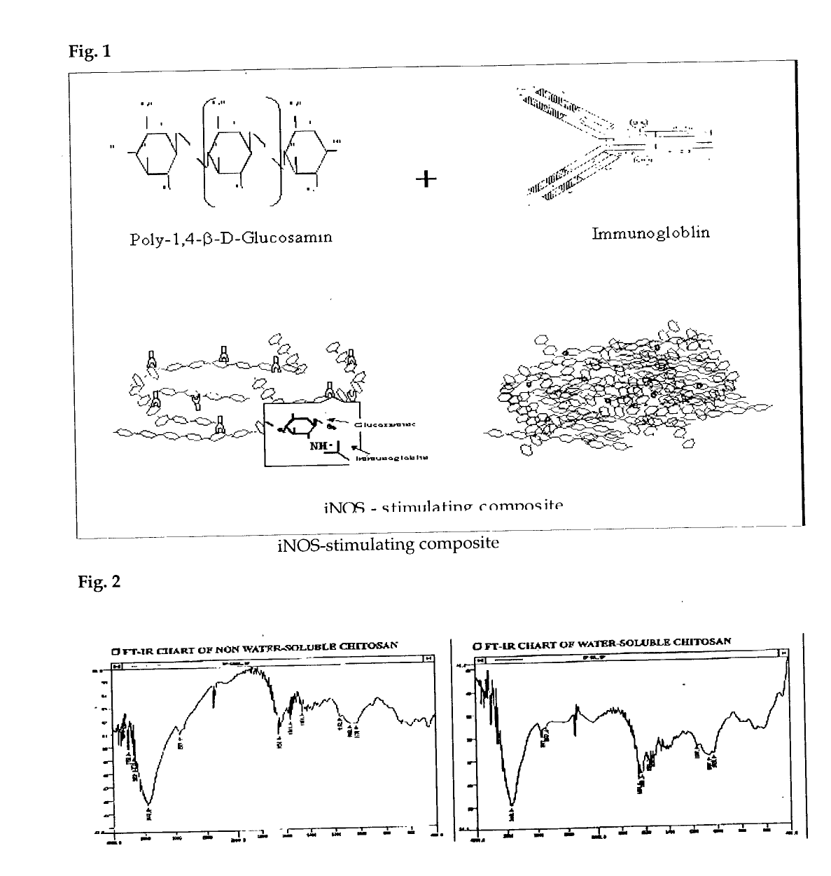

More than once ion-exchange treatment was performed with the filtrate to remove positive charges (NH₂) and introduce negative charges (Cl⁻) to obtain water soluble β-glucosamin fiber, which was further nano-bonded and coated with 10% of immuno-globulin using 1% of cholic acid preparation solution containing 10% of collagen as a catalyst The resulting solution was evaporated, pre-cooled and freeze-dried to produce a composite stimulating iNOS enzyme which induce immuno-reactant NO synthesis (FIG. 1).

### EXPERIMENTAL EXAMPLE 1

10 Peaks of chitin at 1650 cm⁻¹ and chitosan at 1540 cm⁻were identified and the composite obtained in Example 1 showed water soluble characteristic by binding hydroxyl group (OH⁻) with H⁺ of amide group (NH₂) resulted from the hydrogen bonding between unstable functional group and water (H OH) due to repulsion between acetyl group (CH₃C═O) and amide group (NH₂)(FIG. 2).

### EXPERIMENTAL EXAMPLE 2

The total water soluble dietary fiber contained in the composite obtained in Example 1 was measured as follows.

A sample to be analyzed was dried (freeze-dry or dry under the pressure), incubated with enzymes (α-amylase, β-glucanase, protease, amyloglucosidase). The enzyme digestate was then treated with ethanol to precipitate soluble fiber. The precipitate was filtered through the filter paper previously matted with celite, and then contents of protein and ash were determined. The content of the dietary fiber was calculated by employing equation 1 and equation 2(AOAC official method, 16^{th}, 1995),

Blank test value (B, mg)=Blank test average weight of the residue (mg)×PB×AB×100 Equation 1

wherein PB is Blank test protein weight (mg); and AB is Blank test ash weight (mg).

Total dietary fiber (%)=(average weight of residue/average weight of sample)×P×A×B×100 Equation 2

wherein P is protein weigh (mg); A is ash weight (mg); and B is Blank test value.

Total dietary fiber content was higher than 90-99.6% of which water soluble dietary fiber was higher than 80-90% and the purity after recrystallization was higher than 99%.

A degree of deacetylation of chitosan was determined by Potassium polyvinylsulfate solution titration (PVSK, Maeda, M., H. Murakami, H. Ohta, and M. Tajima(1992) Biosci. Biotech.Biochem., 56, 427-431) and more than 80% was deaceylated.

### EXPERIMENTAL EXAMPLE 3

The production of NO was investigated as the following experiment

Macrophage RAW264.7(ATCC) was cultivated in RPMI-1640 medium (RPMI-1640 GibcoBRL 23400-021) 1.62%, sodium hydrogen carbonate 0.2%, penicillin and streptomycin 1%) supplemented with 10% fetal bovine serum (GibcoBRL 26140-079, FBS). Cell was incubated at 37° C., 5% CO₂, and 95% of humidified air (CO₂ incubator).

The production of NO was determined by employing microplate assay and the production of nitrate (NO₃⁻) which is an oxidized product of NO was also determined Macrophage RAW264.7(3×105 cells/ml) was grown in Dulbecco's modified eagle's medium supplemented with 100 U/ml penicillin, 100 μg/ml streptomycin, 10% FBS, 6 g/L HEPES, 3.7 g/L sodium hydrogen carbonate. Cell was stimulated against various control groups. Medium itself, rIFN-γ+the composite of the present invention, rIFN-γ+the composite of the present invention+LPS were stimulated to measure the production of nitrate (NO₃⁻) with time. After macrophage was cultured with INFγ 10 U/ml for 6 hours, the cell was incubated with each of the composite of the present invention (1 μg/ml), LPS(10 μg/ml), and the composite of the present invention (1 μg/ml)+LPS(10 μg/ml) to induce the production of nitrate (NO₃⁻).

The medium was centrifuged (1000 ppm, 10 minutes) and 100 μl of the culture supernatant was reacted with 100 μl of griess reagent (37.5 mM sulphanilic acid, 12.5 mM N-(1-naphthyl) ethylenediamine dihydrochloride, 6.5 mM hydrogen chloride) at room temperature for 10 minutes. Absorbance was measured at 540 nm using spectrophotometer. After correlation coefficient between concentration of NO₃⁻ and absorbance was determined using nitric acid salts having concentration of 0 to 5 μM, the concentration of NO₃⁻-produced from macrophage RAW264.7 was calculated. The result was summarized in Table 1.

TABLE 1

| rIFN-γ | LPS(lipopolysaccharide) | Composite (1 μg/ml) | NO₃_{⁻}(μM) |
|---|---|---|---|
| − | − | +0 h | <5.0 |
| + | − | +12 h | 43.6 ± 4.2 |
| + | − | +24 h | 51.7 ± 3.4 |
| + | − | +48 h | 50.9 ± 1.5 |
| + | + | +24 h | 55.1 ± 3.2 |

−: not added/+: added

TABLE 2
NO production of TAXOL ®

| rIFN-γ | LPS(lipopolysaccharide) | TAXOL ® (10 μg/ml) | NO (μM) |
|---|---|---|---|
| + | − | +0 h | <4.0 |
| + | − | +12 h | 30.56 ± 3.2 |
| + | − | +24 h | 40.7 ± 2.4 |
| + | − | +48 h | 40.8 ± 3.5 |
| + | + | +24 h | 45.1 ± 1.2 |

−: not added/+: added

As shown in Table 1 and Table 2, 56 μg/ml of NO was produced with 1 μg/ml of the composite of the present invention, while 46 μg/ml was with 10 μg/ml of TAXOL®, anticancer agent, which is a control group. NO production with 1 μg/ml of the composite of the present invention was 10 times higher than that with 10 μg/ml of TAXOL®.

It was noted from Experimental Examples and experiment of administration to animals that the composite prepared in Example produced iNOS, eventually produced NO. The composite of the present invention stimulated NK cell proliferation and increased TNF-α cell's activity. The production of NO was increased with higher than 200,000 of molecular weight and greater tan 10 μg/ml of concentration. Thus, it attacks virus, inflammation, tumor, pathogenic bacteria, etc. and accelerates the production of tumor necrosis factor which destroys tumor cells. The increase production of NO provides excellent effects on peripheral vascular expansion and angina pectoris and myocardial infraction because it increase amount of blood passing to the heart due to relaxation of vascular smooth muscles (the NO production was determined with the iNOS enzyme production).

In addition, it has been reported that the glucan absorbs and controls only Cl⁻ ions in the body but it is noted that the composite of this invention even controls NaCl according to the drug delivery experiment after oral administration. The composite of the invention stimulated macrophage and a large quantity of NO was produced. NO having high reactivity was naturally produced for lipopolysaccharide (LPS) The composite of the invention has excellent activities against tumor, virus, and bacteria. When macrophage was stimulated, it was made from L-arginine an amino acid by iNOS.

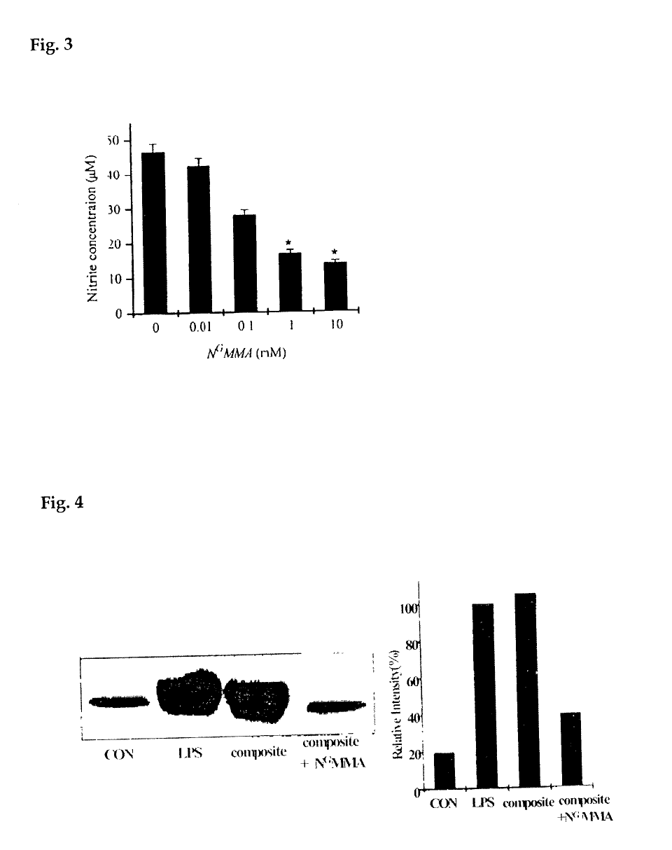

To investigate the mechanism of NO production by a composite prepared by the Example of this invention, macrophage RAW264.7 was incubated with rIFN-γ and N^{G}MMA for 6 hours. The increase production of NO from rIFN-γ-stimulated RAW 264.7 macrophages was decreased by the treatment with N-monomethyl-L-arginine (FIG. 3).

A western blot analysis was performed using anti-iNOS. FIG. 4 represented activities of rIFN-γ and the composite of the invention when macrophage RAW 264.7 was stimulated, each creates NO composite by induced NOS enzyme. Even though it was not showing the data, the composite of the invention involved in the expression of iNOS, and the expression of iNOS was increased by the synergy effect with rIFN-γ or rIFN-γ plus LPS. The increase production of NO from stimulated RAW 264.7 macrophages by the composite was decreased by the treatment with N-monomethyl-L-arginine.

### EXPERIMENTAL EXAMPLE 4

To investigate the expression of TNF-α from a composite prepared by the Example of this invention, the following tests were performed:

Macrophage RAW 264.7 (3×10⁵ cells/well) was cultivated in RPMI-1640 medium supplemented with each of rIFN-γ, composite of this invention, and rIFN-γ+LPS, and rIFN-γ+composite of this invention for 28 hrs.

The secretion amount of TNF-α in the aforementioned cell was measured at a wavelength of 405 nm using ELISA method. Prior to analysis, several drops of 0.05% PBS (phosphate-buffered saline) was added to Tween 20 for its washing. All specimens for analysis was cultivated at 37° C. for 2 hrs. Recombinant murine TNF-α, so diluted, was used as a standard solution. An assay plate was exposed to (containing biotylate murine TNF-α, avidine peroxide, 30% H₂O₂), a substrate solution of ABTS (Azinobis(3-ethylbenzothiazoline-6-sulfonic acid).

TABLE 3

| | | The composite | |
|---|---|---|---|
| rIFN-γ | LPS(lipopolysaccharide) | (10 μg/ml) | TNF-a (ng/ml) |
| − | − | + | 0.81 ± 0.02 |
| + | − | − | 0.76 ± 0.02 |
| + | − | + | 1.34 ± 0.09 |
| + | + | − | 2.80 ± 0.20 |
| + | + | + | 2.92 ± 0.11 |

−: not added/+:added

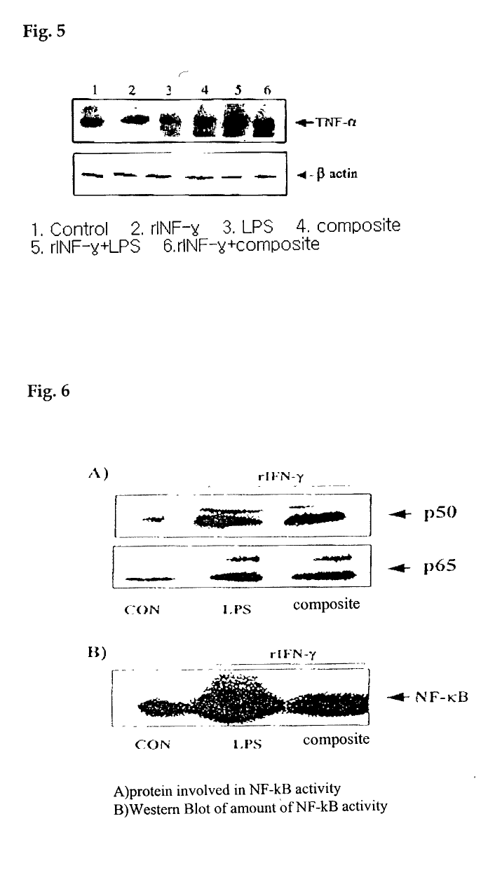

As noted in FIG. 5 and table 3 above, it revealed that when macrophage RAW 264.7 was cultivated in RPMI-1640 medium supplemented with a single composite for 28 hrs, less amount of TNF-α was expressed, but the concomitant use of both rIFN-γ and composite of this invention expressed a large amount of TNF-α.

### EXPERIMENTAL EXAMPLE 5

To investigate the activity of NF-kB from a composite prepared by the Example of this invention, the following tests were performed:

After macrophage RAW 264.7 (5×10⁶ cell/well) and rIFN-γ were cultivated for 6 hrs, they were stimulated by the composite and LPS for 12 hrs.

To produce the lysate of all cells, macrophage RAW 264.7 was boiled with a sample buffered solution [62.5 mM Tris-Cl, pH 6.8, 2% sodium dodecyl sulfate (SDS), 20% glycerol, 10% 2-mercaptoethanol]. The cell-dissolved proteins were separated by 10% SDS-PAGE and transferred to a nitrocellulose paper. This membrane, PBS-tween-20 containing 10% defatted oil, was blocked at room temperature for 1 hr. Then, the membranes was cultivated with anti-iNOS, TNF-α and NF-kB (nucleic factor cover-B) antibodies.

The activity of NF-kB activity was measured by Western blotting. As noted in FIG. 6-A, macrophage, so treated with rIFN-γ and stimulated with LPS, respectively, was found to increase the amount of synthesized protein NF-kB (p65, p50). It also demonstrated that a single composite prepared by Example was in part involved in the generation of synthesized protein NF-kB, but it was stimulated by recombinant rIFN-γ, the activity of NF-kB was increased. Further, as for the activity of NF-kB induced by rIFN-γ from macrophage RAW 264.7, the effect of the composite was confirmed by EMSA electrophoretic mobility shift assay (EMSA).

After being treated with each medium of rIFN-γ+LPS, rIFN-γ+composite of this invention, macrophage RAW 264.7 was cultivated and nucleic extraction was made from each medium. The macrophase was cultivated with ³²P-labelled oligonucleotide having the binding site to NF-kB. Specific The binding capacity to NF-kB could be tested at lane and when the macrophage was treated with rIFN-γ and composite of this invention, DNA-binding capacity was increased (FIG. 6-B).

### EXPERIMENTAL EXAMPLE 6

To evaluate the anti-oxidative effect of the composite prepared by Example of this invention, the anti-oxidative activity was calculated as UV blocking value using the typical antioxidant vitamin C as control, as shown in the following table 3. The anti-oxidative activity of the composite of this invention was measured at the wavelength of 528 nm using an UV-visible spectrometer (Cary, Virian Co., USA).

TABLE 4

| | Composite % solution | | | |
|---|---|---|---|---|
| Category | 0.1% | 0.5% | 1% | 2% |
| Vitamin C | 21.23% | 28.51% | 47.83% | 58.28% |
| Composite | 16.24% | 24.25% | 32.51% | 54.45% |

As noted in the table 4, it revealed that the composite of this invention was an effective antioxidant, since its UV blocking capacity was similar to vitamin C, in view of the fact that UV value of vitamin C is applied as the typical anti-oxidation standards.

### EXPERIMENTAL EXAMPLE 7

According to this invention, there were four types of composites prepared by Example of this invention having the molecular weight of 300,000 Da, 500,000 Da, 750,000 Da and 1,000,000 Da, respectively. Their various properties, such as cytotoxicity, toxicity related to mucosal membrane and vascular system and primary skin irritation, were evaluated as follows:

(1) Cytotoxicity: The widely used MTT analysis and neutral red uptake assay was applied to evaluate the cytotoxicity of the composite and as a result, a high cell viability in skin fibroblast and keratinocyte was observed, irrespective of molecular weight; thus, the composite of this invention showed little cytotoxicity.

(2) Toxicity related to mucosal membrane and vascular system: As a result of HET-CAM analysis, any toxicity reactions (e.g., bleeding, cytolysis, or coagulation) were not observed up to 1.0% solution of the composite, irrespective of molecular weight.

(3) Primary skin irritation: As a result of a 24-hour patch test, any skin irritation was not observed in 1.0% aqueous solution of chitosan with 0.63-1.25 of mean skin score, irrespective of molecular weight.

The example of the mean skin score was to compare the relative percentage of both conventional cosmetics containing organosynthetic preservative (P-M, Germal) mainly and the composite of this invention, when these cosmetics were applied to 20 women in the 20s.

The composite of this invention showed the actual mean skin score with less 40-50%, thus reflecting that it has less skin irritation as a natural product. When the composite in powder form was applied to skin, any skin irritation was not observed.

(4) Antimicrobial and anti-oxidative activities: The paper disk based test showed that the composite of this invention had an excellent preservation capacity as a natural cosmetic.

### EXPERIMENTAL EXAMPLE 8

20 mice each (weighing 50-55 g), irrespective of sex, was repeatedly given the composite prepared in Example of this invention in dosage forms of solution (0.001-0.1%) and powder based on the Korean Pharmacopoeia, so as to perform acute toxicity tests, subcutaneous tests, pyrogen tests, hemolytic tests, and tissue transplantation tests.

As a result, the composite of this invention conformed to the standards. When the composite of this invention was given in an injectable form, compared with control treated with saline solution, pyrexia and sudden death in animal models were not observed with the following results:

Cytotoxicity tests demonstrated that the composite of this invention was not toxic;

When the composite of this invention in aqueous solution was injected to human blood, the counts of leukocyte and their shape changes were not observed;

The coagulation of leukocyte due to invasion of foreign materials were not observed, and

Since the counts of platelet and erythrocyte were not changed, the composite of this invention was excellent in blood profiles.

Cell dyn 900®[Dupont, USA] and Neubauer Chamber and optical microscope were used for these tests.

### EXPERIMENTAL EXAMPLE 9

The biochemical properties of the composite prepared by Example of this invention was evaluated depending each molecular weight, using both inorganic analyses (atom analysis, Beilstein test, ESCA) and organic analyses (¹³C NMR, ¹H NMR, and FI-IR).

To evaluate its biological activity as agricultural chemical, the composite was in vivo applied to 3 pathogens (Phytophthora infestans, Puccinia recondita, and Erysiphe graminis f. sp. Hordei) raising serious problems at home and abroad, at different concentrations of 2, 10, 50, 250 and 1,000 ppm. The higher concentration, the higher antimicrobial activity was observed, and the composite selectively exhibited its high activity against Puccinia recondita.

0.001-3% of the composite (about 10 cc) in aqueous solution, so prepared, was applied at the intervals of 48 hrs 3˜7 times, to the root of tomato-planting soil infected with Enysiphe cichoracerum, leveillula taurica, and Powdery mildew in a tomato vinyl house. When the composite was applied to the root of tomato after three times, the disease was eliminated.

### EXPERIMENTAL EXAMPLE 10

| | |
|---|---|
| Composite of this invention | 0.1 g |
| Lactose | 0.7 g |
| Crystalline cellulose | 0.15 g |
| Magnesium stearate | 0.05 g |
| Total | 1 g |

The above components were pulverized for mixing to produce a tablet form by a direct tableting method. The total amount of each tablets was 100 mg and the content of active ingredients was 1 mg.

### EXPERIMENTAL EXAMPLE 11

| | |
|---|---|
| Composite of this invention | 0.1 g |
| Corn starch | 0.5 g |
| Carboxy cellulose | 0.4 g |
| Total | 1 g |

The above components were pulverized for mixing to produce a powder form. Capsule preparations were made by filling 100 mg of powder into a hard capsule.

### EXPERIMENTAL EXAMPLE 12

The animal experiments were conducted using the composite of both tablets and capsules, so prepared by Experimental examples 10 and 11.

After 20 Sprague-Dawley rats (weighing 300-320 g) were acclimatized with standard feeds for one week, animals were divided into two groups; a test substance group and a control; control was given a high fat diet, while the test group was given once daily a feed mixed with 5% of the composite of this invention ad libitum for 4 weeks at the daily dosing of 2.5 mg. Then, the bloods were collected from both groups.

After 28-day treatment, the total cholesterol and glucose levels was reduced to 15-30% and 15-25%, respectively. The defecation amount was increased up to 25-30% with its five-fold size larger than other materials with low molecular weight.

The composite of this invention is advantageous in that (1) it is effective in preventing adult diseases (such as obesity, constipation, cholesterol), while showing the sustained decomposite differences due to controlled release effect, (2) it helps absorb a drug in the body, and (3) its alcohol decomposite capacity is superior to that of oligosaccharide, a low molecule by more than 5%. Thus, it clearly demonstrated that the composite of this invention may be effectively used in immunology, medical science and nutrition as a natural product in the prevention of diseases.

### EXPERIMENTAL EXAMPLE 13

Since the composite of this invention contains glucosamine having a high affinity in the body with non-toxic effect, its application to the cells can enhance the disease-combat effect. In this context, the inventor et al. tested the wound healing effect of the composite to investigate the cell regeneration in addition to macrophage activity. The composite of this invention was lyophilized to manufacture a disk type, while a wound was induced to rats.

The test group designed to evaluate the wound healing effect and cell regenerated of the composite itself was employed, together with fibronectin as cell adhesion protein with better would healing effect and its modified recombinant protein.

When the composite was applied to a wound site, it was slowly dissolved to form a shallow membrane, while exerting its would healing effect, rapid cell-regeneration effect with viscosity only. The concurrent use of the composite with a wound healing agent demonstrated the most excellent wound healing effect by exchanging a disk once every 3 days. The single use of disk was effective in the treatment of wound and during the concomitant use, the amount of a wound healing agent could be significantly reduced.

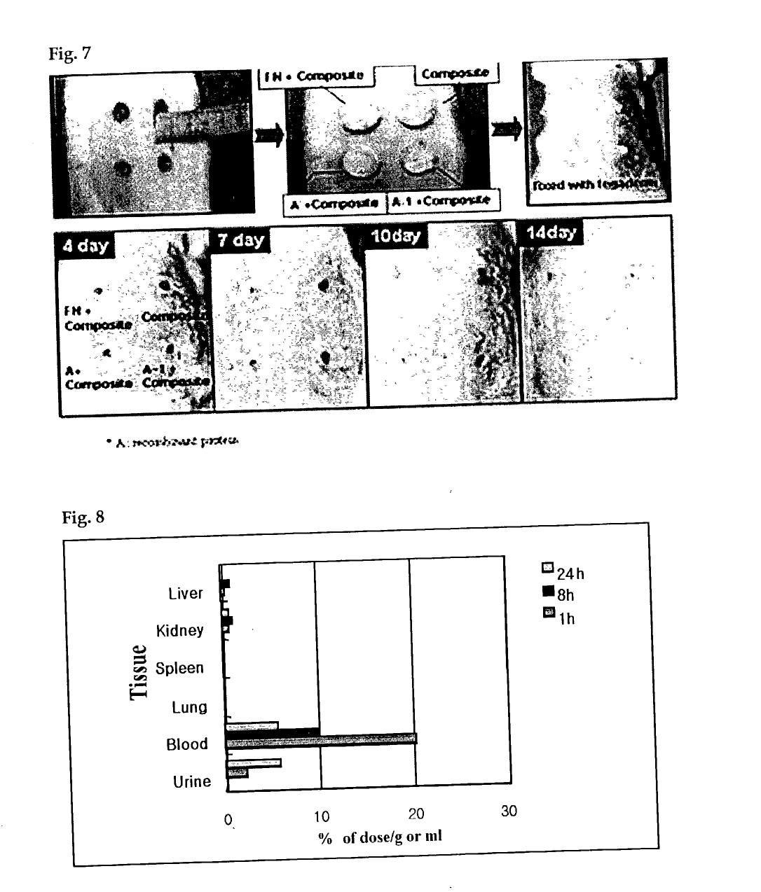

As shown in FIG. 7, it was found that the composite of this invention and its components may be used as a cell regenerator having a high cell affinity and its concurrent use of other drugs may also ensure the excellent synergic effect due to controlled release property [FIG. 7].

### EXPERIMENTAL EXAMPLE 14

1% solution of the composite prepared by Example of this invention was inoculated to bacteria (3.77×10⁶ cfu/g) and fungi (1.2×10⁵ cfu/g) to measure their proliferated counts, compared with control (non-inoculation). In a period ranging from 7 days to 28 days after the commencement of inoculation, their counts remained unchanged and pathogenic bacteria was not detected (0 cfu/g). Accordingly, it was found that the composite of this invention had its own preservative function and antimicrobial activity.

### EXPERIMENTAL EXAMPLE 15

The figures to indicate the digestion or absorption rate during foods intake is expressed by a digestive absorption rate or transition rate in the body. This measurement is commonly made on feces containing nitrogen (protein), carbohydrate, fat, ash, mineral, etc.

The composite of this invention was orally administered to animals, but it was not detected in the serum. The incomplete absorption of the composite in the body is hypothesized that after being cleaved by enzyme in the body, it is transited (transformed) or absorbed, or excreted in a combined form with lipid before going up the stomach.

Since the digestive absorption rate indicates how each nutrient is effectively utilized during foods intake per animal, the digestive absorption rate of each nutrient may be not only changed depending on the blending ratio of foods and cooking, but also affected by health, work and foods intake of animals.

The digestive absorption rate was calculated by the following equations 3and 4.

Digestive absorption rate to the eye=(amount of absorbed ingredient/amount of ingredient in food intake)×100=[(amount of ingredient in food intake−amount of excreted ingredient)/amount of ingredient in food intake]×100 Equation 3

Actual digestive absorption rate=[{amount of ingredient in food intake−amount of excreted ingredient-endogenous lost amount)/amount of ingredient in food intake]}×100 Equation 4

As a result, it was found that the digestive absorption rate of the composite was in the range of 20-40%, indicating a similar level to the general water-soluble edible fiber. To evaluate the vascular transition rate of the composite, normal rats (weighing 200 g) were fasted for 24 hours and 2.5 mg/ml of fluorescent material (FITC; fluorescein isothiocyanate)-composite mixture was injected via tail veins. At the intervals of 1, 8, and 24 hrs, animals were anesthetized to remove the bloods and tissues. As a result of analyzing the detected amount, the vascular transition rate was 98.1% (FIG. 8).

### EXPERIMENTAL EXAMPLE 16

The sustained release tablet represents the sustained effect of drug. To evaluate the production ability and immunology of animals, various composites prepared by Example of this invention (molecular weights ranging from less than 20,000 to 1,000,000) were administered to animals (hens and mice: 20 animals each) in a feed mixed with 0.1% three times at the interval of 7 days compared with control taking the conventional feed under the same conditions, as shown in the following table.

Indeed, breeders in a hen-raising farm (Kangnam Farm located at Pyongtaek City, Kyonggi Province, Korea) observed that hens in the molting season regained their vitality by the general observation.

When the composite of this invention was additionally used, the actual expenditures were economically reduced in various parts. Further, the hardness of white and color of yellow in eggs treated with the composite of this invention was 12-14° without coloring agent, compared with 6-7° in common eggs.

TABLE 5

| | | AMW less | AMW | AMW | AMW | AMW | AMW |
|---|---|---|---|---|---|---|---|
| Item | control | than 20,000 | 100,000 | 200,000 | 300,000 | 600,000 | 1,000,000 |
| production | 0 | No | 2-4 days | 3-5 days | 4-5 days | 4-5 days | 5-6 days |
| ability of hen | | production | | | | | |
| production | 0 | No | 6-7 days | 7 days | 7-8 days | 8-10 days | 8-10 days |
| ability of | | production | | | | | |
| mouse | | | | | | | |

The oral form of the composite was administered to a pig-breeding farming village (Hyungjai Farm located in Yongin, Kyonggi Province, Korea) where virus-associated disease (Picornaviridae Aphthovirus. Food and mouth disease) occurred actually.

20 pigs (70 kg/body weight) in a pigpen showing no virus infection was given the composite in 200 mg tablet three times daily, while an aqueous solution with less than 3% of the composite was frequently administered to drinking water and feeds. As a result, pigs did not show any characteristic symptoms associated with the virus infections without further progression. It indicated that the composite of this invention was indirectly involved in the immune system, apoptosis, antiviral activity, gene modification and metabolism disorder.

### EXAMPLE 17

### Drinking Composite Containing iNOS-Stimulating Composite

75.5 g of refined water was heated up to 60° C. and then, the composite prepared by Example of this invention (2 g), bee honey (3 g), L-tyrosine HCl (0.001 g), vitamin B1 (0.002 g), vitamin B₂ (0.01 g), nicotinic acid amide (0.01 g), citric acid (0.2 g), fructose (15 g), pantothenic acid calcium (0.01 g), vitamin B6 (0.003 g), and shell extract (2.4 )g was added to the refined water and stirred for dissolution. The mixing solution was filled to various bottles with packaging units of 250,500 and 1000 ml and sterilized at less than 90° C. for 20 mins.

As described above, the composite stimulating iNOS enzyme which induces immuno-reactant NO synthesis of the present invention improves the movement of food/fluid through the intestinal tract, lowers cholesterol and fat levels and provides excellent functional activities. Further it can be used widely as food additives unlike the conventional fibers and as oriental medicine ingredients. Therefore, the composite of the invention is being considered as an excellent resource for products of the future to be free from harmful pollution from artificial compositions, from fear of mutation derivation, and unnecessary expectation of genetic variation technology.

What is claimed is:

1. A process for preparing a composite stimulating iNOS enzyme which induces immuno-reactant NO synthesis comprising the steps of:
1) adjusting water soluble chitin/chitosan in 10-40% NaCl solution to be pH between 4.5 to 8.5 and performing ultrasonic degradation;
2) performing lysozyme degradation and then ion-exchange;
3) performing ion-exchange with fractions of step 2) to produce β-glucosamin fiber; and
4) nano-coating and binding the β-glucosamin fiber of step 3) with 0.1-15% of immuno-protein.

2. The process for preparing a composite stimulating iNOS enzyme which induces immuno-reactant NO synthesis according to claim 1, wherein said ultrasonic degradation is performed for from 1 to 14 hours at 10to 80° C. of temperature and 10 to 80 KHz of wavelength.

3. The process for preparing a composite stimulating iNOS enzyme which induces immuno-reactant NO synthesis according to claim 1, wherein said ion-exchange in step 2) is cation-exchange and then anion-exchange and polydispersity efficiency is in the range of from 1.1 to 1.9.

4. The process for preparing a composite stimulating iNOS enzyme which induces immuno-reactant NO synthesis according to claim 1, wherein said nano-coating and binding with immuno-protein in step 4) is performed in the presence of collagen and cholic acid as a catalyst.

5. A composite stimulating iNOS enzyme which induces immuno-reactant NO synthesis prepared by the process in claim 1, having 100,000 to 1,000,000 of molecular weight.

6. A pharmaceutical composition containing said composite prepared by the process in claim 1.

7. A food additive containing said composite prepared by the process in claim 1.
